(19) United States
(12) Patent Application Publication
(10) Pub. No.: US 2006/0093677 A1
(43) Pub. Date: May 4, 2006

(54) Methods for making pharmaceutical formulations comprising deagglomerated microparticles

(76) Inventors: Donald E. Chickering III, Framingham, MA (US); Shaina Reese, Arlington, MA (US); Sridhar Narasimhan, Framingham, MA (US); Julie A. Straub, Winchester, MA (US); Howard Bernstein, Cambridge, MA (US); David Altreuter, Brookline, MA (US); Eric K. Huang, Waltham, MA (US)

Correspondence Address: SUTHERLAND ASBILL & BRENNAN LLP, 999 PEACHTREE STREET, N.E., ATLANTA, GA, 30309, US

(21) Appl. No.: 11/305,461

(22) Filed: Dec. 16, 2005

Related U.S. Application Data
(62) Division of application No. 10/324,558, filed on Dec. 19, 2002.

(51) Int. Cl. A61K 9/14

(52) U.S. Cl. 424/489

(57) ABSTRACT

Methods are provided for making a dry powder blend pharmaceutical formulation comprising (i) forming microparticles which comprise a pharmaceutical agent; (ii) providing at least one excipient in the form of particles having a volume average diameter that is greater than the volume average diameter of the microparticles; (iii) blending the microparticles with the excipient to form a powder blend; and (iv) jet milling the powder blend to deagglomerate at least a portion of any of the microparticles which have agglomerated, while substantially maintaining the size and morphology of the individual microparticles. Jet milling advantageously can eliminate the need for more complicated wet deagglomeration processes, can lower residual moisture and solvent levels in the microparticles (which leads to better stability and handling properties for dry powder formulations), and can improve wettability, suspendability, and content uniformity of dry powder blend formulations.

### CROSS-REFERENCE TO RELATED APPLICATIONS

This is a divisional of U.S. application Ser. No. 10/324,558, filed Dec. 19, 2002, now pending. That application is incorporated herein by reference in its entirety.

### BACKGROUND OF THE INVENTION

This invention is generally in the field of compositions comprising microparticles, and more particularly to methods of producing microparticulate formulations for the delivery of pharmaceutical materials, such as drugs and diagnostic agents, to patients.

Microencapsulation of therapeutic and diagnostic agents is known to be a useful tool for enhancing the controlled delivery of such agents to humans or animals. For these applications, microparticles having very specific sizes and size ranges are needed in order to effectively deliver these agents. Microparticles, however, may tend to agglomerate during their production and processing, thereby undesirably altering the effective size of the particles, to the detriment of the microparticle formulation's performance and/or reproducibility. Agglomeration depends on a variety of factors, including, but not limited to, the temperature, humidity, and compaction forces to which the microparticles are exposed, as well as the particular materials and methods used in making the microparticles. It therefore would be useful to deagglomerate the microparticles post production and/or the microparticle dry powder formulations using a process that does not substantially affect the size and morphology of the microparticle as originally formed. Such a process preferably should be simple and operate at ambient conditions to minimize equipment and operating costs and to avoid degradation of pharmaceutical agents, such as thermally labile drugs.

Microparticle production techniques typically require the use of one or more aqueous or organic solvents. For example, an organic solvent can be combined with, and then removed from, a polymeric matrix material in the process of forming polymeric microparticles by spray drying. An undesirable consequence, however, is that the microparticles often retain solvent residue. It is highly desirable to minimize these solvent residue levels in pharmaceutical formulations. It therefore would be advantageous to develop a process that enhances solvent removal from microparticle formulations.

Similarly, it would be desirable to reduce moisture levels in microparticle formulations, irrespective of the source by which the moisture is introduced, in order to decrease caking, increase flowability, and improve storage stability of the formulation. For example, an aqueous solvent can be used to dissolve or disperse an excipient to facilitate mixing of the excipient with microparticles, after which the aqueous solvent is removed. It therefore would be advantageous to develop a process that enhances moisture removal from microparticle formulations.

Excipients often are added to the microparticles and pharmaceutical agents in order to provide the microparticle formulations with certain desirable properties or to enhance processing of the microparticle formulations. For example, the excipients can facilitate administration of the microparticles, minimize microparticle agglomeration upon storage or upon reconstitution, facilitate appropriate release or retention of the active agent, and/or enhance shelf life of the product. Representative types of these excipients include osmotic agents, bulking agents, surfactants, preservatives, wetting agents, pharmaceutically acceptable carriers, diluents, binders, disintegrants, glidants, and lubricants. It is important that the process of combining these excipients and microparticles yield a uniform blend. Combining these excipients with the microparticles can complicate production and scale-up; it is not a trivial matter to make such microparticle pharmaceutical formulations, particularly on a commercial scale.

Laboratory scale methods for producing microparticle pharmaceutical formulations may require several steps, which may not be readily or efficiently transferred to larger scale production. Examples of these steps include dispersing the microparticles, size classification of the microparticles, drying and/or lyophilizing them, loading them with one or more active agents, and combining them with one or more excipient materials to form a homogenous product ready for packaging. Some process steps such as freezing the microparticles (e.g., as part of a solvent removal process) by the use of liquid nitrogen are expensive and difficult to execute in a clean room for large volume operations. Other process steps, such as sonication, may require expensive custom made equipment to perform on larger scales. It would be advantageous to develop pharmaceutical formulation production methods to eliminate, combine, or simplify any of these steps.

It therefore would be desirable to provide deagglomerated microparticle pharmaceutical formulations having low residuals. It would be particularly desirable for dry forms of the microparticle formulation to disperse and suspend well upon reconstitution, providing an injectable formulation. It would be desirable for dry forms of the microparticle formulation to disperse well in the dry form, providing an inhalable formulation. It would be desirable for dry forms of the microparticle formulation to disperse well upon oral administration, providing a solid oral dosage form.

It would be desirable to provide a method for both deagglomerating microparticulate pharmaceutical formulations and reducing residual moisture (and/or solvent) levels in these formulations, using a process that does not substantially affect the size and morphology of the microparticle as originally formed. It would also be desirable to provide methods for making uniform blends of deagglomerated microparticles and excipients, preferably without the use of an excipient solvent. Such methods desirably would be adaptable for efficient, commercial scale production.

### SUMMARY OF THE INVENTION

Methods are provided for making a dry powder pharmaceutical formulation comprising (i) forming microparticles which comprise a pharmaceutical agent; (ii) providing at least one excipient (e.g., a bulking agent, surface active agent, wetting agent, or osmotic agent) in the form of particles having a volume average diameter that is greater than the volume average diameter of the microparticles; (iii) blending the microparticles with the excipient to form a powder blend; and (iv) jet milling the powder blend to deagglomerate at least a portion of any of the microparticles which have agglomerated, while substantially maintaining the size and morphology of the individual microparticles.

The excipient particles can have, for example, a volume average size between 10 and 500 μm, between 20 and 200 μm, or between 40 and 100 μm, depending in part on the particular pharmaceutical formulation and route of administration. Examples of excipients include, but are not limited to, lipids, sugars, amino acids, and polyoxyethylene sorbitan fatty acid esters, and combinations thereof. In one embodiment, the excipient is selected from the group consisting of lactose, mannitol, sorbitol, trehalose, xylitol, and combinations thereof. In another embodiment, the excipient comprises hydrophobic amino acids such as leucine, isoleucine, alanine, glycine, valine, proline, cysteine, methionine, phenylalanine, or tryptophan. In another embodiment, the excipient comprises binders, disintegrants, glidants, diluents, coloring agents, flavoring agents, sweeteners, and lubricants for a solid oral dosage formulation such as for a tablet, capsule, or wafer. Two or more different excipients can be blended with the microparticles, in one or more steps. In one embodiment, the microparticles consist essentially of a therapeutic or prophylactic pharmaceutical agent. In another embodiment, the microparticles further comprises a shell material (e.g., a polymer, protein, lipid, sugar, or amino acid).

In another aspect, a method is provided for making a dry powder blend pharmaceutical formulation comprising two or more different pharmaceutical agents. In one method, the steps include (a) providing a first quantity of microparticles which comprise a first pharmaceutical agent; (b) providing a second quantity of microparticles which comprise a second pharmaceutical agent; (c) blending the first quantity and the second quantity to form a powder blend; and (d) jet milling the powder blend to deagglomerate at least a portion of any of the microparticles which have agglomerated, while substantially maintaining the size and morphology of the individual microparticles. This method can further comprise blending an excipient material with the first quantity, the second quantity, the powder blend, or a combination thereof.

In yet another embodiment, a method is provided for making pharmaceutical formulations comprising microparticles, wherein the method comprises (i) spraying an emulsion, solution, or suspension which comprises a solvent and a pharmaceutical agent through an atomizer to form droplets of the solvent and the pharmaceutical agent; (ii) evaporating a portion of the solvent to solidify the droplets and form microparticles; and (iii) jet milling the microparticles to deagglomerate at least a portion of agglomerated microparticles, if any, while substantially maintaining the size and morphology of the individual microparticles. In one embodiment, the microparticles consist essentially of a therapeutic or prophylactic pharmaceutical agent. In another embodiment, the emulsion, solution, or suspension further comprises a shell material (e.g., a polymer, lipid, sugar, protein, or amino acid).

In a further embodiment, a method is provided for making pharmaceutical formulations comprising microparticles, wherein the method comprises: (i) forming microparticles which comprise a pharmaceutical agent and a shell material; and jet milling the microparticles to deagglomerate at least a portion of any of the microparticles which have agglomerated, while substantially maintaining the size and morphology of the individual microparticles. Spray drying or other methods can be used in the microparticle-forming step. In one embodiment, the pharmaceutical agent is dispersed throughout the shell material. In another embodiment, the microparticles comprise a core of the pharmaceutical agent, which is surrounded by the shell material. Examples of shell materials include, but are not limited to, polymers, amino acids, sugars, proteins, carbohydrates, and lipids. In one embodiment, the shell material comprises a biocompatible synthetic polymer.

In another embodiment, jet milling is used to increase the percent crystallinity or decrease amorphous content of the drug within the microparticles.

In one embodiment of these methods, the jet milling is performed with a feed gas and/or grinding gas supplied to the jet mill at a temperature of less than about 80° C., more preferably less than about 30° C. In one embodiment, the feed gas and/or grinding gas supplied to jet mill consists essentially of dry nitrogen gas.

In various embodiments of these methods, the microparticles have a number average size between 1 and 10 μm, have a volume average size between 2 and 50 μm, and/or have an aerodynamic diameter between 1 and 50 μm.

In one embodiment, the microparticles comprise microspheres having voids or pores therein. In a preferred variation of this embodiment, the pharmaceutical agent is a therapeutic or prophylactic agent, which is hydrophobic.

In one embodiment of these methods, the pharmaceutical agent is a therapeutic or prophylactic agent. Examples of classes of these agents include non-steroidal anti-inflammatory agents, corticosteroids, anti-neoplastics, anti-microbial agents, anti-virals, anti-bacterial agents, anti-fungals, anti-asthmatics, bronchiodilators, antihistamines, immunosuppressive agents, anti-anxiety agents, sedatives/hypnotics, anti-psychotic agents, anticonvulsants, and calcium channel blockers. Examples of therapeutic or prophylactic agents include celecoxib, rofecoxib, docetaxel, paclitaxel, acyclovir, alprazolam, amiodaron, amoxicillin, anagrelide, bactrim, beclomethasone dipropionate, biaxin, budesonide, bulsulfan, carbamazepine, ceftazidime, cefprozil, ciprofloxcin, clarithromycin, clozapine, cyclosporine, estradiol, etodolac, famciclovir, fenofibrate, fexofenadine, fluticasone propionate, gemcitabine, ganciclovir, itraconazole, lamotrigine, loratidine, lorazepam, meloxicam, mesalamine, minocycline, nabumetone, nelfinavir, mesylate, olanzapine, oxcarbazepine, phenytoin, propfol, ritinavir, SN-38, sulfasalazine, tracrolimus, tiagabine, tizanidine, valsartan, voriconazole, zafirlukast, zilueton, and ziprasidone.

In another embodiment, the pharmaceutical agent is a diagnostic agent, such as an ultrasound contrast agent.

Dry powder pharmaceutical formulations are also provided. These formulations comprise blended or unblended microparticles that have been deagglomerated as described herein, which may provide reduced moisture content and residual solvent levels in the formulation, improved suspendability of the formulation, improved aerodynamic properties, decreased amorphous drug content, and (for blends) improved content uniformity.

### BRIEF DESCRIPTION OF THE DRAWINGS

FIG. 1 is a process flow diagram of a preferred process for producing deagglomerated microparticle formulations.

FIG. 2 illustrates a diagram of a typical jet mill useful in the method of deagglomerating microparticles.

FIGS. 3A-B are SEM images of microspheres taken before and after jet milling.

### DETAILED DESCRIPTION OF THE INVENTION

Improved methods have been developed for making pharmaceutical formulations comprising deagglomerated microparticles and for making blends of microparticles and excipients that have enhanced content uniformity. Jet milling advantageously breaks up microparticle agglomerates. The reduction of microparticle agglomerates leads to improved suspendability for injectable dosage forms, improved dispersibility for oral dosage forms, or improved aerodynamic properties for inhalable dosage forms. Moreover, jet milling beneficially lowers residual moisture and solvent levels in the microparticles, leading to better stability and handling properties for the dry powder pharmaceutical formulations.

I. The Microparticle Formulations

The formulations include microparticles comprising one or more pharmaceutical agents such as a therapeutic or a diagnostic agent, and optionally one or more excipients. In one embodiment, the formulation is a uniform dry powder blend comprising microparticles of a pharmaceutical agent blended with larger microparticles of an excipient.

A. Microparticles

As used herein, the term “microparticle” includes microspheres and microcapsules, as well as microparticles, unless otherwise specified. Microparticles may or may not be spherical in shape. Microcapsules are defined as microparticles having an outer shell surrounding a core of another material, in this case, the pharmaceutical agent. The core can be gas, liquid, gel, or solid. Microspheres can be solid spheres, can be porous and include a sponge-like or honeycomb structure formed by pores or voids in a matrix material or shell, or can include a single internal void in a matrix material or shell.

In one embodiment, the microparticle is formed entirely of the pharmaceutical agent. In another embodiment, the microparticle has a core of pharmaceutical agent encapsulated in a shell. In another embodiment, the pharmaceutical agent is interspersed within the shell or matrix. In another embodiment, the pharmaceutical agent is uniformly mixed within the material comprising the shell or matrix. Optionally, the microparticles can be blended with one or more excipients.

1. Size and Morphology

As used herein, the terms “size” or “diameter” in reference to microparticles refers to the number average particle size, unless otherwise specified. An example of an equation that can be used to describe the number average particle size is shown below:

$$\frac{{\sum }_{i = 1}^{p} {n}_{i} {d}_{i}}{{\sum }_{i = 1}^{p} {n}_{i}}$$

where n=number of particles of a given diameter (d).

As used herein, the term “volume average diameter” refers to the volume weighted diameter average. An example of an equation that can be used to describe the volume average diameter is shown below:

$${[ \frac{{\sum }_{i = 1}^{p} {n}_{i} {d}_{i}^{3}}{{\sum }_{i = 1}^{p} {n}_{i}} ]}^{1 / 3}$$

where n=number of particles of a given diameter (d).

As used herein, the term “aerodynamic diameter” refers to the equivalent diameter of a sphere with density of 1 g/mL were it to fall under gravity with the same velocity as the particle analyzed. The values of the aerodynamic average diameter for the distribution of particles are reported. Aerodynamic diameters can be determined on the dry powder using an Aerosizer (TSI), which is a time of flight technique, or by cascade impaction, or liquid impinger techniques.

Particle size analysis can be performed on a Coulter counter, by light microscopy, scanning electron microscopy, transmission electron microscopy, laser diffraction methods, light scattering methods or time of flight methods. Where a Coulter method is described, the powder is dispersed in an electrolyte, and the resulting suspension analyzed using a Coulter Multisizer II fitted with a 50-μm aperture tube.

The jet milling process described herein deagglomerates agglomerated microparticles, such that the size and morphology of the individual microparticles is substantially maintained. That is, a comparison of the microparticle size before and after jet milling should show a volume average size reduction of at least 15% and a number average size reduction of no more than 75%.

In the formulations, the microparticles preferably have a number average size between about 1 and 20 μm. It is believed that the jet milling processes will be most usefuil in deagglomerating microparticles having a volume average diameter or aerodynamic average diameter greater than about 2 μm. In one embodiment, the microparticles have a volume average size between 2 and 50 μm. In another embodiment, the microparticles have an aerodynamic diameter between 1 and 50 μm.

The microparticles are manufactured to have a size (i.e., diameter) suitable for the intended route of administration. Particle size also can affect RES uptake. For intravascular administration, the microparticles preferably have a number average diameter of between 0.5 and 8 μm. For subcutaneous or intramuscular administration, the microparticles preferably have a number average diameter of between about 1 and 100 μm. For oral administration for delivery to the gastrointestinal tract and for application to other lumens or mucosal surfaces (e.g., rectal, vaginal, buccal, or nasal), the microparticles preferably have a number average diameter of between 0.5 μm and 5 mm. A preferred size for administration to the pulmonary system is an aerodynamic diameter of between 1 and 5 μm, with an actual volume average diameter (or an aerodynamic average diameter) of 5 μm or less.

In one embodiment, the microparticles comprise microspheres having voids therein. In one embodiment, the microspheres have a number average size between 1 and 3 μm and a volume average size between 3 and 8 μm.

In another embodiment, jet milling increases the crystallinity or decreases the amorphous content of the drug within the microspheres as assessed by differential scanning calorimetry.

2. Pharmaceutical Agents

The pharmaceutical agent is a therapeutic, diagnostic, or prophylactic agent. The pharmaceutical agent is sometimes referred to herein generally as a “drug” or “active agent.” The pharmaceutical agent may be present in an amorphous state, a crystalline state, or a mixture thereof. The pharmaceutical agent may be labeled with a detectable label such as a fluorescent label, radioactive label or an enzymatic or chromatographically detectable agent.

A wide variety of therapeutic, diagnostic and prophylactic agents can be loaded into the microparticles. These can be proteins or peptides, sugars, oligosaccharides, nucleic acid molecules, or other synthetic or natural agents. Representative examples of suitable drugs include, but are not limited to, the following categories and examples of drugs and alternative forms of these drugs such as alternative salt forms, free acid forms, free base forms, and hydrates:
analiesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, and meprobamate);
antiasthmatics (e.g., ketotifen and traxanox);
antibiotics (e.g., neomycin, streptomycin, chloramphenicol, cephalosporin, ampicillin, penicillin, tetracycline, and ciprofloxacin);
antidepressants (e.g., nefopam, oxypertine, doxepin, amoxapine, trazodone, amitriptyline, maprotiline, phenelzine, desipramine, nortriptyline, tranylcypromine, fluoxetine, imipramine, imipramihe pamoate, isocarboxazid, trimipramine, and protriptyline);
antidiabetics (e.g., biguanides and sulfonylurea derivatives);
antifungal agents (e.g., griseofulvin, ketoconazole, itraconizole, virconazole, amphotericin B, nystatin, and candicidin);
antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);
anti-inflammatories (e.g., (non-steroidal) celecoxib, rofecoxib, indomethacin, ketoprofen, flurbiprofen, naproxen, ibuprofen, ramifenazone, piroxicam, (steroidal) cortisone, dexamethasone, fluazacort, hydrocortisone, prednisolone, and prednisone);
antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan);
antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);
immunosuppressive azents (e.g., cyclosporine, azathioprine, mizoribine, and FK506 (tacrolimus), sirolimus);
antimigraine agents (e.g., ergotamine, propanolol, and dichloralphenazone);
sedatives/hypnotics (e.g., barbiturates such as pentobarbital, pentobarbital, and secobarbital; and benzodiazapines such as flurazepam hydrochloride, and triazolam);
antianginal agents (e.g., beta-adrenergic blockers; calcium channel blockers such as nifedipine, and diltiazem; and nitrates such as nitroglycerin, and erythrityl tetranitrate);
antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, lithium citrate, prochlorperazine, aripiprazole, and risperdione);
antimanic agents (e.g., lithium carbonate);
antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, flecainide acetate, tocainide, and lidocaine);
antiarthritic azents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium);
antigout agents (e.g., colchicine, and allopurinol);
anticoagulants (e.g., heparin, heparin sodium, and warfarin sodium);
thrombolytic agents (e.g., urokinase, streptokinase, and alteplase);
antifibrinolytic agents (e.g., aminocaproic acid);
hemorheologic agents (e.g., pentoxifylline);
antiplatelet agents (e.g., aspirin);
anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, paramethadione, ethotoin, phenacemide, secobarbitol sodium, clorazepate dipotassium, oxcarbazepine and trimethadione);
antiparkinson agents (e.g., ethosuximide);
antihistamines/antipruritics (e.g., hydroxyzine, diphenhydramine, chlorpheniramine, brompheniramine maleate, cyproheptadine hydrochloride, terfenadine, clemastine fumarate, azatadine, tripelennamine, dexchlorpheniramine maleate, methdilazine);
agents useful for calcium regulation (e.g., calcitonin, and parathyroid hormone);
antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, clarithromycin and colistin sulfate);
antiviral agents (e.g., interferons, zidovudine, amantadine hydrochloride, ribavirin, and acyclovir);
antimicrobials (e.g., cephalosporins such as ceftazidime; penicillins; erythromycins; and tetracyclines such as tetracycline hydrochloride, doxycycline hyclate, and minocycline hydrochloride, azithromycin, clarithromycin);
anti-infectives (e.g., GM-CSF);
bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; salbutarnol; ipratropium bromide; ketotifen; salmeterol; xinafoate; terbutaline sulfate; theophylline; nedocromil sodium; metaproterenol sulfate; albuterol);
inhalant corticosteroids (e.g., beclomethasone dipropionate (BDP), beclomethasone dipropionate monohydrate; budesonide, triamcinolone; flunisolide; fluticasone proprionate; mometasone);
steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);
hypoglycemic agents (e.g., human insulin, purified beef insulin, purified pork insulin, glyburide, chlorpropamide, glipizide, tolbutamide, and tolazamide);
hypolipidemic agents (e.g., clofibrate, dextrothyroxine sodium, probucol, pravastitin, atorvastatin, lovastatin, and niacin);
proteins (e.g., DNase, alginase, superoxide dismutase, and lipase);
nucleic acids (e.g., sense or anti-sense nucleic acids encoding any therapeutically useful protein, including any of the proteins described herein);
agents useful for erythropoiesis stimulation (e.g., erythropoietin);
antiulcer/antireflux agents (e.g., famotidine, cimetidine, and ranitidine hydrochloride);
antinauseants/antiemetics (e.g., meclizine hydrochloride, nabilone, prochlorperazine, dimenhydrinate, promethazine hydrochloride, thiethylperazine, and scopolamine);
oil-soluble vitamins (e.g., vitamins A, D, E, K, and the like); as well as other drugs such as mitotane, halonitrosoureas, anthrocyclines, and ellipticine. A description of these and other classes of useful drugs and a listing of species within each class can be found in Martindale, The Extra Pharmacopoeia, 30th Ed. (The Pharmaceutical Press, London 1993).

Examples of other drugs useful in the compositions and methods described herein include ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol ftumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered water-soluble.

Preferred drugs include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine flumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

In one embodiment, the pharmaceutical agent is a hydrophobic compound, particularly a hydrophobic therapeutic agent. Examples of such hydrophobic drugs include, but are not limited to, celecoxib, rofecoxib, paclitaxel, docetaxel, acyclovir, alprazolam, amiodaron, amoxicillin, anagrelide, bactrim, biaxin, budesonide, bulsulfan, carbamazepine, ceftazidime, cefprozil, ciprofloxicin, clarithromycin, clozapine, cyclosporine, diazepam, estradiol, etodolac, famciclovir, fenofibrate, fexofenadine, gemcitabine, ganciclovir, itraconazole, lamotrigine, loratidine, lorazepam, meloxicam, mesalamine, minocycline, modafinil, nabumetone, nelfinavir mesylate, olanzapine, oxcarbazepine, phenytoin, propofol, ritinavir, SN-38, sulfamethoxazol, sulfasalazine, tracrolimus, tiagabine, tizanidine, trimethoprim, valium, valsartan, voriconazole, zafirlukast, zileuton, and ziprasidone. In this embodiment, the microparticles preferably are porous.

In one embodiment, the pharmaceutical agent is for pulmonary administration. Examples include, but are not limited to, corticosteroids such as budesonide, fluticasone propionate, beclomethasone dipropionate, mometasone, flunisolide, and triamcinolone acetonide, other steroids such as testosterone, progesterone, and estradiol, leukotriene inhibitors such as zafirlukast and zileuton, antibiotics such as cefprozil, amoxicillin, antifungals such as ciprofloxacin, and itraconazole, bronchiodilators such as albuterol, formoterol, and salmeterol, antineoplastics such as paclitaxel and docetaxel, and peptides or proteins such as insulin, calcitonin, leuprolide, granulocyte colony-stimulating factor, parathyroid hormone-related peptide, and somatostatin.

In another embodiment, the pharmaceutical agent is a contrast agent for diagnostic imaging, particularly a gas for ultrasound imaging. In a preferred embodiment, the gas is a biocompatible or pharmacologically acceptable fluorinated gas, as described, for example, in U.S. Pat. No. 5,611,344 to Bernstein et al., which is incorporated herein by reference. The term “gas” refers to any compound that is a gas or capable of forming a gas at the temperature at which imaging is being performed. The gas may be composed of a single compound or a mixture of compounds. Perfluorocarbon gases are preferred; examples include CF₄, C₂F₆, C₃F₈, C₄F₁₀, SF₆, C₂F₄, and C₃F₆. Other imaging agents can be incorporated in place of a gas, or in combination with the gas. Imaging agents that may be utilized include commercially available agents used in positron emission tomography (PET), computer assisted tomography (CAT), single photon emission computerized tomography, x-ray, fluoroscopy, and magnetic resonance imaging (MRI). Microparticles loaded with these agents can be detected using standard techniques available in the art and commercially available equipment. Examples of suitable materials for use as contrast agents in MRI include the gadolinium chelates currently available, such as diethylene triamine pentacetic acid (DTPA) and gadopentotate dimeglumine, as well as iron, magnesium, manganese, copper and chromium. Examples of materials useful for CAT and x-rays include iodine based materials for intravenous administration, such as ionic monomers typified by diatrizoate and iothalamate, non-ionic monomers such as iopamidol, isohexol, and ioversol, non-ionic dimers, such as iotrol and iodixanol, and ionic dimers, e.g., ioxagalte. Other useful materials include barium for oral use.

3. The Shell Material

The shell material can be a synthetic material or a natural material. The shell material can be water soluble or water insoluble. The microparticles can be formed of non-biodegradable or biodegradable materials, although biodegradable materials are preferred, particularly for parenteral administration. Examples of types of shell materials include, but are not limited to, polymers, amino acids, sugars, proteins, carbohydrates, and lipids. Polymeric shell materials can be degradable or non-degradable, erodible or non-erodible, natural or synthetic. Non-erodible polymers may be used for oral administration. In general, synthetic polymers are preferred due to more reproducible synthesis and degradation. Natural polymers also may be used. Natural biopolymers that degrade by hydrolysis, such as polyhydroxybutyrate, may be of particular interest. The polymer is selected based on a variety of performance factors, including the time required for in vivo stability, i.e., the time required for distribution to the site where delivery is desired, and the time desired for delivery. Other selection factors may include shelf life, degradation rate, mechanical properties, and glass transition temperature of the polymer.

Representative synthetic polymers are poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and copolymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellubse, cellulose acetate, cellulose propionate, cellulose acetate butyrate, cellulose acetate phthalate, carboxyethyl cellulose, cellulose triacetate, and cellulose sulphate sodium salt jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl. acrylate), and poly(octadecyl acrylate) Oointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin and prolamines, for example, zein, and polysaccharides such as alginate, cellulose and polyhydroxyalkanoates, for example, polyhydroxybutyrate. The in vivo stability of the matrix can be adjusted during the production by using polymers such as polylactide-co-glycolide copolymerized with polyethylene glycol (PEG). PEG, if exposed on the external surface, may extend the time these materials circulate post intravascular administration, as it is hydrophilic and has been demonstrated to mask RES (reticuloendothelial system) recognition.

Examples of preferred non-biodegradable polymers include ethylene vinyl acetate, poly(meth)acrylic acid, polyamides, copolymers and mixtures thereof.

Bioadhesive polymers can be of particular interest for use in targeting of mucosal surfaces (e.g., in the gastrointestinal tract, mouth, nasal cavity, lung, vagina, and eye). Examples of these include polyanhydrides, polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Representative amino acids that can be used in the shell include both naturally occurring and non-naturally occurring amino acids. The amino acids can be hydrophobic or hydrophilic and may be D amino acids, L amino acids or racemic mixtures. Amino acids that can be used include, but are not limited to, glycine, arginine, histidine, threonine, asparagine, aspartic acid, serine, glutamate, proline, cysteine, methionine, valine, leucine, isoleucine, tryptophan, phenylalanine, tyrosine, lysine, alanine, and glutamine. The amino acid can be used as a bulking agent, or as an anti-crystallization agent for drugs in the amorphous state, or as a crystal growth inhibitor for drugs in the crystalline state or as a wetting agent. Hydrophobic amino acids such as leucine, isoleucine, alanine, glycine, valine, proline, cysteine, methionine, phenylalanine, tryptophan are more likely to be effective as anticrystallization agents or crystal growth inhibitors. In addition, amino acids can serve to make the shell have a pH dependency that can be used to influence the pharmaceutical properties of the shell such as solubility, rate of dissolution or wetting.

The shell material can be the same or different from the excipient material, if present. In one embodiment, the excipient can comprise the same classes or types of material used to form the shell. In another embodiment, the excipient comprises one or more materials different from the shell material. In this latter embodiment, the excipient can be a surfactant, wetting agent, salt, bulking agent, etc. In one embodiment, the formulation comprises (a) microparticles that have a core of a drug and a shell comprising a sugar or amino acid, blended with (b) another sugar or amino acid that functions as a bulking or tonicity agent.

B. Excipients

The term “excipient” refers to any non-active ingredient of the formulation intended to facilitate delivery and administration by the intended route. For example, the excipient can comprise proteins, amino acids, sugars or other carbohydrates, starches, lipids, or combinations thereof. The excipient may enhance handling, stability, aerodynamic properties, and dispersibility of the active agent.

In preferred embodiments, the excipient is a dry powder (e.g., in the form of microparticles,) which is blended with drug microparticles. Preferably, the excipient microparticles are larger in size than the pharmaceutical microparticles. In one embodiment, the excipient microparticles have a volume average size between about 10 and 500 μm, preferably between 20 and 200 μm, more preferably between 40 and 100 μm.

Representative amino acids that can be used in the drug matrices include both naturally occurring and non-naturally occurring amino acids. The amino acids can be hydrophobic or hydrophilic and may be D amino acids, L amino acids or racemic mixtures. Amino acids which can be used include, but are not limited to: glycine, arginine, histidine, threonine, asparagine, aspartic acid, serine, glutamate, proline, cysteine, methionine, valine, leucine, isoleucine, tryptophan, phenylalanine, tyrosine, lysine, alanine, glutamine. The amino acid can be used as a bulking agent, as a wetting agent, or as a crystal growth inhibitor for drugs in the crystalline state. Hydrophobic amino acids such as leucine, isoleucine, alanine, glycine, valine, proline, cysteine, methionine, phenylalanine, or tryptophan are more likely to be effective as crystal growth inhibitors. In addition, amino acids can serve to make the matrix have a pH dependency that can be used to influence the pharmaceutical properties of the matrix, such as solubility, rate of dissolution, or wetting.

Examples of excipients include pharmaceutically acceptable carriers and bulking agents, including sugars such as lactose, mannitol, trehalose, xylitol, sorbitol, dextran, sucrose, and fructose. These sugars may also serve as wetting agents. Other suitable excipients include surface active agents, dispersants, osmotic agents, binders, disintegrants, glidants, diluents, color agents, flavoring agents, sweeteners, and lubricants. Examples include sodium desoxycholate; sodium dodecylsulfate; polyoxyethylene sorbitan fatty acid esters, e.g., polyoxyethylene 20 sorbitan monolaurate (TWEEN™ 20), polyoxyethylene 4 sorbitan monolaurate (TWEEN™ 21), polyoxyethylene 20 sorbitan monopalmitate (TWEEN™ 40), polyoxyethylene 20 sorbitan monooleate (TWEEN™ 80); polyoxyethylene alkyl ethers, e.g., polyoxyethylele 4 lauryl ether (BRIJ™ 30), polyoxyethylene 23 lauryl ether (BRIJ™ 35), polyoxyethylene 10 oleyl ether (BRIJ™ 97); polyoxyethylene glycol esters, e.g., poloxyethylene 8 stearate (MYRJ™ 45), poloxyethylene 40 stearate (MYRJ™ 52); Tyloxapol; Spans; and mixtures thereof.

Examples of binders include starch, gelatin, sugars, gums, polyethylene glycol, ethylcellulose, waxes and polyvinylpyrrolidone. Examples of disintegrants (including super disintegrants) includes starch, clay, celluloses, croscarmelose, crospovidone and sodium starch glycolate. Examples of glidants include colloidal silicon dioxide and talc. Examples of diluents include dicalcium phosphate, calcium sulfate, lactose, cellulose, kaolin, mannitol, sodium chloride, dry starch and powdered sugar. Examples of lubricants include talc, magnesium stearate, calcium stearate, stearic acid, hydrogenated vegetable oils, and polyethylene glycol.

The amounts of excipient for a particular formulation depend on a variety of factors and can be selected by one skilled in the art. Examples of these factors include the choice of excipient, the type and amount of drug, the microparticle size and morphology, and the desired properties and route of administration of the final formulation.

In one embodiment for injectable microparticles, a combination of mannitol and TWEEN™ 80 is blended with polymeric microspheres. In one case, the mannitol is provided at between 100 and 200% w/w, preferably 130 and 170% w/w, microparticles, while the TWEEN™ 80 is provided at between 0.1 and 10% w/w, preferably 3.0 and 5.1% w/w microparticles. In another case, the mannitol is provided with a volume average particle size between 10 and 500 μm.

In another embodiment, the excipient comprises binders, disintegrants, glidants, diluents, color agents, flavoring agents, sweeteners, lubricants, or combinations thereof for use in a solid oral dosage form. Examples of solid oral dosage forms include capsules, tablets, and wafers.

II. Methods of Making the Microparticle Formulations

The pharmaceutical formulations are made by a process that includes forming a quantity of microparticles comprising a pharmaceutical agent and having a selected size and morphology; and then jet milling the microparticles effective to deagglomerate the agglomerated microparticles while substantially maintaining the size and morphology of the individual microparticles. That is, the jet milling step deagglomerates the microparticles without significantly fracturing individual microparticles. The jet milling step can advantageously reduce moisture content and residual solvent levels in the formulation, can improve the suspendability and wettability of the dry powder formulation (e.g., for better injectability), and give the dry powder formulation improved aerodynamic properties (e.g., for better pulmonary delivery).

In one embodiment, the process further (and optionally) includes blending the microparticles with one or more excipients, to create uniform blends of microparticles and excipients in the dry state. Preferably, the blending is conducted before the jet milling step. If desired, however, some or all of the components of the blended formulation can be jet milled before being blended together. Additionally, such blends can be further jet milled again to deagglomerate the blended microparticles.

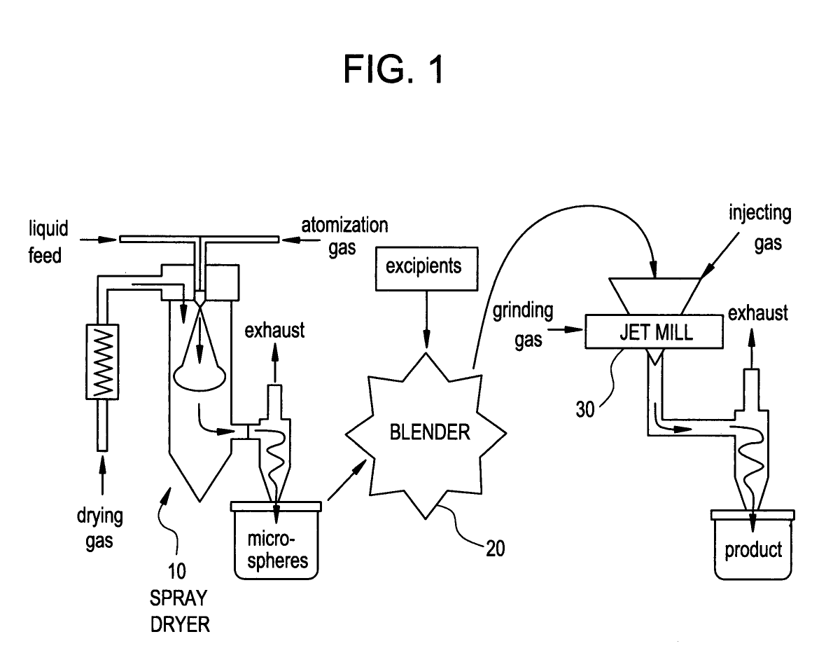

One specific embodiment of the process is illustrated in FIG. 1. In this embodiment, microspheres are produced by spray drying in spray dryer 10. The microspheres are then blended with excipients in blender 20. Finally, the blended microspheres/excipients are fed to jet mill 30, where the microspheres are deagglomerated and residual solvent levels reduced. The moisture level in the microsphere formulation also can be reduced in the jet milling process. In addition, the content uniformity of the blended microspheres/excipients can be improved over that of the non-jet milled blended microspheres/excipients.

The processes described herein generally can be conducted using batch, continuous, or semi batch methods.

Microparticle Production

The microparticles can be made using a variety of techniques known in the art. Suitable techniques include spray drying, melt extrusion, compression molding, fluid bed drying, solvent extraction, hot melt encapsulation, phase inversion encapsulation, and solvent evaporation.

In the most preferred embodiment, the microparticles are produced by spray drying. See, e.g., U.S. Pat. No. 5,853,698 to Straub et al.; U.S. Pat. No. 5,611,344 to Bernstein et al.; U.S. Pat. No. 6,395,300 to Straub et al.; and U.S. Pat. No. 6,223,455 to Chickering III et al., which are incorporated herein by reference. For example, the microparticles can be produced by dissolving a pharmaceutical agent and/or shell material in an appropriate solvent, (and optionally dispersing a solid or liquid active agent, pore forming agent (e.g., a volatile salt), or other additive into the solution containing the pharmaceutical agent and/or shell material) and then spray drying the solution, to form microparticles. As defined herein, the process of “spray drying” a solution containing a pharmaceutical agent and/or shell material refers to a process wherein the solution is atomized to form a fine mist and dried by direct contact with hot carrier gases. Using spray drying equipment available in the art, the solution containing the pharmaceutical agent and/or shell material may be atomized into a drying chamber, dried within the chamber, and then collected via a cyclone at the outlet of the chamber. Representative examples of types of suitable atomization devices include ultrasonic, pressure feed, air atomizing, and rotating disk. The temperature may be varied depending on the solvent or materials used. The temperature of the inlet and outlet ports can be controlled to produce the desired products. The size of the particulates of pharmaceutical agent and/or shell material is a function of the nozzle used to spray the solution of pharmaceutical agent and/or shell material, nozzle pressure, the solution and atomization flow rates, the pharmaceutical agent and/or shell material used, the concentration of the pharmaceutical agent and/or shell material, the type of solvent, the temperature of spraying (both inlet and outlet temperature), and the molecular weight of a shell material such as a polymer or other matrix material. Generally, the higher the molecular weight, the larger the particle size, assuming the concentration is the same (because an increase in molecular weight generally increases the solution viscosity). Microparticles having a target diameter between 0.5 and 500 μm can be obtained. The morphology of these microparticles depends, for example, on the selection of shell material, concentration, molecular weight of a shell material such as a polymer or other matrix material, spray flow, and drying conditions.

Solvent evaporation is described by Mathiowitz et al., J. Scanning Microscopy, 4:329 (1990); Beck et al., Fertil. Steril, 31:545 (1979); and Benita et al., J. Pharm. Sci., 73:1721 (1984), the teachings of which are incorporated herein. In this method, a shell material is dissolved in a volatile organic solvent such as methylene chloride. A pore forming agent as a solid or as a liquid may be added to the solution. The pharmaceutical agent can be added as either a solid or solution to the shell material solution. The mixture is sonicated or homogenized and the resulting dispersion or emulsion is added to an aqueous solution that may contain a surface active agent (such as TWEEN™20, TWEEN™80, polyethylene glycol, or polyvinyl alcohol), and homogenized to form an emulsion. The resulting emulsion is stirred until most of the organic solvent evaporates, leaving microparticles. Several different polymer concentrations can be used (e.g., 0.05-0.60 g/mL). Microparticles with different sizes (1-1000 μm) and morphologies can be obtained by this method. This method is particularly useful for shell materials comprising relatively stable polymers such as polyesters.

Hot-melt microencapsulation is described in Mathiowitz et al., Reactive Polymers, 6:275 (1987), the teachings of which are incorporated herein. In this method, a shell material is first melted and then mixed with a solid or liquid pharmaceutical agent. A pore forming agent as a solid or in solution may be added to the melt. The mixture is suspended in a non-miscible solvent (e.g., silicon oil), and, while stirring continuously, heated to 5° C. above the melting point of the shell material. Once the emulsion is stabilized, it is cooled until the shell material particles solidify. The resulting microparticles are washed by decantation with a shell material non-solvent, such as petroleum ether, to give a free-flowing powder. Generally, microparticles with sizes between 50 and 5000 μm are obtained with this method. The external surfaces of particles prepared with this technique are usually smooth and dense. This procedure is used to prepare microparticles made of polyesters and polyanhydrides. However, this method is limited to shell materials such as polymers with molecular weights between 1000 and 50,000. Preferred polyanhydrides include polyanhydrides made of biscarboxyphenoxypropane and sebacic acid with molar ratio of 20:80 (P(CPP-SA) 20:80) (MW 20,000) and poly(fumaric-co-sebacic) (20:80) (MW 15,000).

Solvent removal is a technique primarily designed for shell materials such as polyanhydrides. In this method, the solid or liquid pharmaceutical agent is dispersed or dissolved in a solution of a shell material in a volatile organic solvent, such as methylene chloride. This mixture is suspended by stirring in an organic oil (e.g., silicon oil) to form an emulsion. Unlike solvent evaporation, however, this method can be used to make microparticles from shell materials such as polymers with high melting points and different molecular weights. The external morphology of particles produced with this technique is highly dependent on the type of shell material used.

Extrusion techniques can be used to make microparticles. In this method, microparticles made of shell materials such as gel-type polymers, such as polyphosphazene or polymethylmethacrylate, are produced by dissolving the shell material in an aqueous solution, suspending if desired a pore forming agent in the mixture, homogenizing the mixture, and extruding the material through a microdroplet forming device, producing microdroplets that fall into a slowly stirred hardening bath of an oppositely charged ion or polyelectrolyte solution. The advantage of these systems is the ability to further modify the surface of the hydrogel microparticles by coating them with polycationic polymers, like polylysine, after fabrication. Microparticle size can be controlled by using various size extruders or atomizing devices.

Phase inversion encapsulation is described in U.S. Pat. No. 6,143,211 to Mathiowitz, et al., which is incorporated herein by reference. By using relatively low viscosities and/or relatively low shell material concentrations, by using solvent and nonsolvent pairs that are miscible and by using greater than ten fold excess of nonsolvent, a continuous phase of nonsolvent with dissolved pharmaceutical agent and/or shell material can be rapidly introduced into the nonsolvent. This causes a phase inversion and spontaneous formation of discreet microparticles, typically having an average particle size of between 10 nm and 10 μm.

Jet Milling

As used herein, the terms “jet mill” and “jet milling” include and refer to the use of any type of fluid energy impact mills, including, but not limited to, spiral jet mills, loop jet mills, and fluidized bed jet mills, with or without internal air classifiers. As used herein, jet milling is a technique for substantially deagglomerating microparticle agglomerates that have been produced during or subsequent to formation of the microparticles, by bombarding the feed particles with high velocity air or other gas, typically in a spiral or circular flow. The jet milling process conditions are selected so that the microparticles are substantially deagglomerated while substantially maintaining the size and morphology of the individual microparticles, which can be quantified as providing a volume average size reduction of at least 15% and a number average size reduction of no more than 75%. The process is characterized by the acceleration of particles in a gas stream to high velocities for impingement on other particles, similarly accelerated.

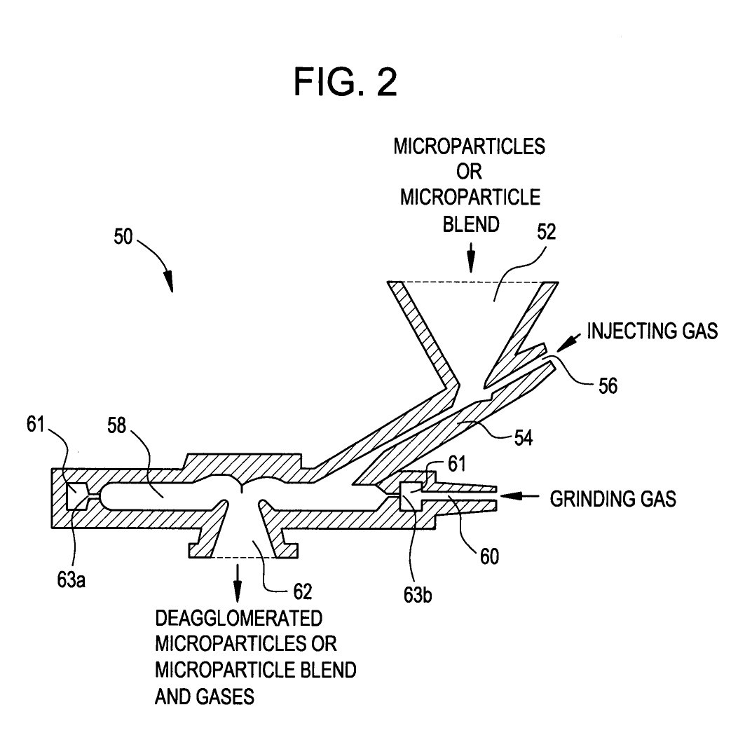

A typical spiral jet mill is illustrated in FIG. 2. The jet mill 50 is shown in cross-section. Microparticles (blended or unblended) are fed into feed chute 52, and injection gas is fed through one or more ports 56. The microparticles are forced through injector 54 into deagglomeration chamber 58. The microparticles enter an extremely rapid vortex in the chamber 58, where they collide with one another and with chamber walls until small enough to be dragged out of a central discharge port 62 in the mill by the gas stream (against centrifugal forces experienced in the vortex). Grinding gas is fed from port 60 into gas supply ring 61. The grinding gas then is fed into the chamber 58 via a plurality of apertures; only two 63a and 63b are shown. Deagglomerated, uniformly blended, microparticles are discharged from the mill 50.

The selection of the material forming the bulk of the microparticles and the temperature of the microparticles in the mill are among the factors that affect deagglomeration. Therefore, the mill optionally can be provided with a temperature control system. For example, the control system may heat the microparticles, rendering the material less brittle and thus less easily fractured in the mill, thereby minimizing unwanted size reduction. Alternatively, the control system may need to cool the microparticles to below the glass transition or melting temperature of the material, so that deagglomeration is possible.

In one embodiment, a hopper and feeder are used to control introduction of dry powder materials into the jet mill, providing a constant flow of material to the mill. Examples of suitable feeders include vibratory feeders and screw feeders. Other means known in the art also can be used for introducing the dry powder materials into the jet mill.

In one operation method, the microparticles are aseptically fed to the jet mill via a feeder, and a suitable gas, preferably dry nitrogen, is used to feed and grind the microparticles through the mill. Grinding and feed gas pressures can be adjusted based on the material characteristics. Preferably, these gas pressures are between 0 and 10 bar, more preferably between 2 and 8 bar. Microparticle throughput depends on the size and capacity of the mill. The milled microparticles can be collected by filtration or, more preferably, cyclone.

It was discovered that jet milling the microparticles not only deagglomerates the microparticles, but also can lower the residual solvent and moisture levels in the microparticles. Thus, a single process step was found to provide both deagglomeration and moisture/solvent reduction. To achieve reduced residual levels, the injection/grinding gas preferably is a low humidity gas, such as dry nitrogen. In one embodiment, the injection/grinding gas is at a temperature less than 100° C. (e.g., less than 75° C., less than 50° C., less than 25° C., etc.).

It was also found that by jet milling the microparticles (or a microparticle-comprising dry powder blend) to deagglomerate them, it improved the dispersibility of the microparticles. As used herein, the term “dispersibility” includes the suspendability of a powder (e.g., a quantity or dose of microparticles) within a liquid, as well as the aerodynamic properties of such a powder or such microparticles. Accordingly, the term “improved dispersibility” refers to a reduction of particle-particle interactions of the microparticles of a powder within a liquid or a gas.

In another embodiment, jet milling the microparticles can induce transformation of the drug within the microparticles from an at least partially amorphous form to a less amorphous form (i.e., a more crystalline form). This advantageously provides the drug in a more stable form.

Blending

In a preferred embodiment, dry uniform microparticle blends are produced. That is, the deagglomerated microparticles can be blended with another material, such as an excipient material, a (second) pharmaceutical agent, or a combination thereof. Jet milling can advantageously enhance the content uniformity of a dry powder blend.

In a preferred embodiment, the excipient or pharmaceutical agent is in the form of a dry powder. In one embodiment, the methods for deagglomerating further include blending microparticles with one or more other materials having a larger particle size than that of the microparticles.

In one embodiment, a blend is made by deagglomerating microparticles comprising a first pharmaceutical agent, and then blending these microparticles (in one or more steps) with one or more excipient materials and with a second pharmaceutical agent. In a second embodiment, a blend is made of two or more pharmaceutical agents, without an excipient material. For example, the method could include deagglomerating microparticles comprising a first pharmaceutical agent, and then blending these microparticles with a second pharmaceutical agent. Alternatively, microparticles comprising the first pharmaceutical agent could be blended with microparticles comprising the second pharmaceutical agent, and the resulting blend could then be deagglomerated.

The blending can be conducted in one or more steps, in a continuous, batch, or semi-batch process. For example, if two or more excipients are used, they can be blended together before, or at the same time as, being blended with the microparticles. Generally, there are two approaches for adding excipients to microparticles: wet addition and dry addition. Wet addition typically involves adding an aqueous solution of the excipient to the microparticles. The microparticles are then dispersed by mixing and may require additional processing such as sonication to fully disperse the microparticles. To create the dry dispersion, the water must be removed, for example, using methods such as lyophilization. It would be desirable to eliminate the wet processing, and thus use dry addition. In dry addition, the excipients are added to the microparticles in the dry state and the components are blended using standard dry, solid mixing techniques. Dry blending advantageously eliminates the need to dissolve or disperse the excipient in a solvent before combining the excipient with the microparticles and thus eliminates the need to subsequently remove that solvent. This is particularly advantageous when the solvent removal step would otherwise require lyophilization, freezing, distillation, or vacuum drying steps.

Content uniformity of solid-solid pharmaceutical blends is critical. Jet milling can be conducted on the microparticles either before and/or after blending, to enhance content uniformity. In a preferred embodiment, the microparticles are blended with one or more excipients of interest, and the resulting blend is then jet milled to yield a uniform mixture of deagglomerated microparticles and excipient.

Jet-milling advantageously can provide improved wetting and dispersibility upon reconstitution. In addition, the resulting microparticle formulation can provide improved injectability, passing through the needle of a syringe more easily.

Jet-milling advantageously can provide improved dispersibility of the dry powder, which provides for improved aerodynamic properties for pulmonary administration.

In another embodiment, the jet-milled microparticles or jet-milled blends of microparticles/excipient can be further processed into a solid oral dosage form, such as a power-filled capsule, a wafer, or a tablet. Jet-milling advantageously can provide improved wetting and dispersibility upon oral dosing as a solid oral dosage form formed from jet-milled microparticles or jet-milled microparticle/excipient blend.

The blending can be carried out using essentially any technique or device suitable for combining the microparticles with one or more other materials (e.g., excipients), preferably to achieve uniformity of blend. For example, the blending process can be performed using a variety of blenders. Representative examples of suitable blenders include V-blenders, slant-cone blenders, cube blenders, bin blenders, static continuous blenders, dynamic continuous blenders, orbital screw blenders, planetary blenders, Forberg blenders, horizontal double-arm blenders, horizontal high intensity mixers, vertical high intensity mixers, stirring vane mixers, twin cone mixers, drum mixers, and tumble blenders. The blender preferably is of a strict sanitary design required for pharmaceutical products.

Tumble blenders are preferred for batch operation. In one embodiment, blending is accomplished by aseptically combining two or more components (which can include both dry components and small portions of liquid components) in a suitable container. The container may, for example, be a polished, stainless steel or a glass container. The container is then sealed and placed (i.e., secured) into the tumble blender (e.g., TURBULA™, distributed by Glen Mills Inc., Clifton, N.J., USA, and made by Willy A. Bachofen A G, Maschinenfabrik, Basel, Switzerland) and then mixed at a specific speed for an appropriate duration. (TURBULA™ lists speeds of 22, 32, 46, 67, and 96 rpm for its model T2F, which has a 2 L basket and a maximum load of 10 kg.) Durations preferably are between about five minutes and six hours, more preferably between about 5 and 60 minutes. Actual operating parameters will depend, for example, on the particular formulation, size of the mixing vessel, and quantity of material being blended.

For continuous or semi-continuous operation, the blender optionally may be provided with a rotary feeder, screw conveyor, or other feeder mechanism for controlled introduction of one or more of the dry powder components into the blender.

Other Steps in the Formulation Process

The blended and jet milled product may undergo additional processing. Representative examples of such processes include lyophilization or vacuum drying to further remove residual solvents, temperature conditioning to anneal materials, size classification to recover or remove certain fractions of the particles (i.e., to optimize the size distribution), compression molding to form a tablet or other geometry, and packaging. In one embodiment, oversized (e.g., 20 μm or larger, preferably 10 μm or larger) microparticles are separated from the microparticles of interest. Some formulations also may undergo sterilization, such as by gamma irradiation.

III. Applications for Using the Microparticle Formulations

In preferred embodiments, the microparticle formulations are administered to a human or animal in need thereof, for the delivery of a therapeutic, diagnostic, or prophylactic agent in an effective amount. The formulations can be administered in dry form or dispersed in a physiological solution for injection or oral administration. The dry form can be aerosolized and inhaled for pulmonary administration. The route of administration depends on the pharmaceutical agent being delivered.

The microparticle formulations containing an encapsulated imaging agent may be used in vascular imaging, as well as in applications to detect liver and renal diseases, in cardiology applications, in detecting and characterizing tumor masses and tissues, and in measuring peripheral blood velocity. The microparticles also can be linked with ligands that minimize tissue adhesion or that target the microparticles to specific regions of the body in vivo as known in the art.

The invention can further be understood with reference to the following non-limiting examples.

### EXAMPLES

Blending and jet milling experiments were carried out, combining PLGA microspheres, TWEEN™ 80 (Spectrum Chemicals, New Brunswick, N.J.), and mannitol (Spectrum Chemicals). TWEEN™ 80 is hereinafter referred to as “Tween80.” Dry blending was carried out based on the following relative amounts of each material: 39 mg of PLGA microspheres, 54.6 mg of mannitol, and 0.16 mg of Tween80.

A TURBULA™ inversion mixer (model: T2F) was used for blending. An Alpine Aeroplex Spiral Jet Mill (model: 50AS), with dry nitrogen gas as the injector and grinding gases, was used for de-agglomeration. Four blending processes were tested, and three different jet mill operating conditions were tested for each of the four blending processes, as described in Examples 1-4.

In all of the studies, the dry powder was fed manually into the jet mill and hence the powder feed rate was not constant. It should be noted that although the powder feeding was manual, the feed rate was calculated to be approximately 1.0 g/min. for all of the studies. Feed rate is the ratio of total material processed in one batch to the total batch time. Particle size measurement of the jet milled samples, unless otherwise indicated, was conducted using a Coulter Multisizer II with a 50 μm aperture. Where aerodynamic particle size is reported, the analysis was performed using an Aerosizer (TSI, Inc.).

The PLGA microspheres used in Examples 1-4 originated from the same batch (“Lot A”). The microspheres were prepared as follows: A polymer emulsion was prepared, composed of droplets of an aqueous phase suspended in a continuous polymer/organic solvent phase. The polymer was a commercially obtained poly(lactide-co-glycolide) (PLGA) (50:50), and the organic solvent was methylene chloride. The resulting emulsion was spray dried at a flow rate of 150 mL/min with an outlet temperature of 12° C. on a custom spray dryer with a drying chamber.

The PLGA microspheres used in Example 5 were from Lot A as described above and from Lot B and Lot C, which were prepared as follows: Lot B: An emulsion was created as for Lot A, except that the polymer was provided from a different commercial source. The resulting emulsion was spray dried at a flow rate of 200 mL/min with an outlet temperature of 12° C. on a custom spray dryer with a drying chamber. Lot C: An emulsion was created in the same manner as for Lot B, except that the resulting emulsion was spray dried at a flow rate of 150 mL/min. Table A below provides information describing the spray drying conditions and bulk microspheres made thereby.

TABLE A
Spray Dried Microspheres and Parameters

| | Liquid | | | Drying Gas | | | |
|---|---|---|---|---|---|---|---|
| | Flow Rate | Atom rate | Inlet | Flow Rate | | | Bulk % |
| Lot ID | (mL/min) | (L/min) | Temp. (° C.) | (Kg/Hr) | Xn (μm) | Xv (μm) | Moisture |
| A | 150 | 115 | 57 | 110 | 2.83 | 8.07 | 6.62% |
| B | 200 | 110 | 55 | 150 | 2.26 | 6.03 | 10.28% |
| C | 150 | 95 | 54 | 110 | 2.60 | 6.15 | 28.60% |

Xn = number mean average diameter
Xv = volume mean average diameter

### Example 1

### Jet Milling of PLGA Microspheres/Excipient Blend (Made by Dry/Dry Two-Step Blending)

Blending was conducted in two dry steps. In the first step, 5.46 g of mannitol and 0.16 g of Tween80 were added into a 125 mL glass jar. The jar was then set in the TURBULA™ mixer for 15 minutes at 46 min⁻¹. In the second step, 3.9 g of PLGA microspheres were added into the glass jar containing the blended mannitol and Tween80. The jar was then set in the TURBULA™ mixer for 30 minutes at 46 min⁻¹. A dry blended powder was produced. The dry blended powder was then fed manually into a jet mill for particle deagglomeration. Three sets of operating conditions for the jet mill were used, as described in Table 1.

TABLE 1
Jet Mill Operating Conditions

| | Injector Gas | Grinding Gas |
|---|---|---|
| Sample | Pressure (bar) | Pressure (bar) |
| 1.1 | 3.9 | 3.0 |
| 1.2 | 3.0 | 2.9 |
| 1.3 | 8.0 | 6.6 |

The resulting jet milled samples were analyzed for particle size. For comparison, a representative sample of mannitol (pre blending and jet milling), and a control sample (blended but not jet milled) were analyzed. The Coulter Multisizer II results are shown in Table 2. The data reported for mannitol are from particle size analysis using a Malvern Mastersizer, because particle size analysis could not be performed using a Coulter Multisizer, due to the aqueous solubility of mannitol.

TABLE 2
Results of Particle Size Analysis

| | Number Avg. | Volume Avg. |
|---|---|---|
| Sample | Particle Size, Xₙ (μm) | Particle Size, Xᵥ (μm) |
| Mannitol | NA | 18.65 |
| Control | 2.64 | 6.92 |
| 1.1 | 2.12 | 5.17 |
| 1.2 | 2.11 | 5.09 |
| 1.3 | 1.96 | 4.07 |

By comparing the data of the control sample and jet milled samples, it can be inferred that the jet milling provides significant particle deagglomeration. As the grinding air pressure was increased, Xₙ stayed nearly constant, but Xᵥ decreased.

### Example 2

### Jet Milling of PLGA Microspheres/Excipient Blend Made by Wet/Dry Two-Step Blending

Blending was conducted in two steps: one wet and one dry. In the first step, mannitol and Tween80 were blended in liquid form. A 500 mL quantity of Tween80/mannitol vehicle was prepared from Tween80, mannitol, and water. The vehicle had concentrations of 0.16 % Tween80 and 54.6 mg/mL mannitol. The vehicle was transferred into a 1200 mL Virtis glass jar and then frozen with liquid nitrogen. The vehicle was frozen as a shell around the inside of the jar in 30 minutes, and then subjected to vacuum drying in a Virtis dryer (model: FreezeMobile 8EL) at 31 mTorr for 115 hours. At the end of vacuum drying, the vehicle was in the form of a powder, believed to be the Tween80 homogeneously dispersed with the mannitol. In the second step, 3.9 g of PLGA microspheres were added into the glass jar containing the blended mannitol and Tween80. The jar was then set in the TURBULA™ mixer for 30 minutes at 46 min⁻¹. A dry blended powder was produced. The dry blended powder was then fed manually into a jet mill for particle deagglomeration. Three sets of operating conditions for the jet mill were used, as described in Table 3.

TABLE 3
Jet Mill Operating Conditions

| | Injector Gas | Grinding Gas |
|---|---|---|
| Sample | Pressure (bar) | Pressure (bar) |
| 2.1 | 3.9 | 3.0 |
| 2.2 | 3.0 | 2.9 |
| 2.3 | 7.4 | 6.2 |

The resulting jet milled samples were analyzed for particle size. For comparison, a control sample (blended but not jet milled) was similarly analyzed. The Coulter Multisizer II results are shown in Table 4.

TABLE 4
Results of Particle Size Analysis

| | Number Avg. | Volume Avg. |
|---|---|---|
| Sample | Particle Size, Xₙ (μm) | Particle Size, Xᵥ (μm) |
| Control | 2.78 | 8.60 |
| 2.1 | 1.98 | 4.52 |
| 2.3 | 1.99 | 4.11 |
| 2.3 | 1.93 | 3.37 |

Again, by comparing the data of the control sample and jet milled samples, it can be inferred that the jet milling provides significant particle deagglomeration.

### Example 3

### Jet Milling of PLGA Microspheres/Excipient Blend Made by One-Step Dry Blending

In an attempt to reduce the blending time even further, a single blending step was tested. First, 5.46 g of mannitol was added into a 125 mL glass jar. Then 0.16 g of Tween80 and 3.9 g of PLGA microspheres were added into the jar. The jar was then set in the TURBULA™ mixer for 30 minutes at 46 min⁻¹. A dry blended powder was produced. The dry blended powder was fed manually into a jet mill for particle deagglomeration. Three sets of operating conditions for the jet mill were used, as described in Table 5.

TABLE 5
Jet Mill Operating Conditions

| | Injector Gas | Grinding Gas |
|---|---|---|
| Sample | Pressure (bar) | Pressure (bar) |
| 3.1 | 3.9 | 3.0 |
| 3.2 | 3.0 | 2.9 |
| 3.3 | 8.0 | 6.6 |

The resulting jet milled samples were analyzed for particle size. For comparison, a control sample (blended but not jet milled) was similarly analyzed. The Coulter Multisizer II values are shown in Table 6.

TABLE 6
Results of Particle Size Analysis

| | Number Avg. | Volume Avg. |
|---|---|---|
| Sample | Particle Size, Xₙ (μm) | Particle Size, Xᵥ (μm) |
| Control | 2.33 | 7.57 |
| 3.1 | 2.08 | 5.47 |
| 3.2 | 2.15 | 5.91 |
| 3.3 | 2.13 | 4.91 |

Again, by comparing the data of the control sample and jet milled samples, it can be inferred that the jet milling provides significant particle deagglomeration.

### Example 4

### Jet Milling of PLGA Microspheres/Excipient Blend (Made by One-Step Dry Blending—Higher Speed)

In an attempt to reduce the blending time even further, a single blending step was tested using an increased blending speed for the TURBULA™ mixer as compared to the speed used in Example 3. First, 5.46 g of mannitol was added into a 125 mL glass jar. Then 0.16 g of Tween80 and 3.9 g of PLGA microspheres were added into the jar. The jar was then set in the TURBULA™ mixer for 30 minutes, with the blending speed was set at 96 min⁻¹. A dry blended powder was produced. The dry blended powder was fed manually into a jet mill for particle deagglomeration. Three sets of operating conditions for the jet mill were used, as described in Table 7.

TABLE 7
Jet Mill Operating Conditions

| | Injector Gas | Grinding Gas |
|---|---|---|
| Sample | Pressure (bar) | Pressure (bar) |
| 4.1 | 3.9 | 3.0 |
| 4.2 | 3.0 | 2.9 |
| 4.3 | 8.0 | 6.6 |

The resulting jet milled samples were analyzed for particle size. For comparison, a control sample (blended but not jet milled) was similarly analyzed. The Coulter Multisizer II results are shown in Table 8.

TABLE 8
Results of Particle Size Analysis

| | Number Avg. | Volume Avg. |
|---|---|---|
| Sample | Particle Size, Xₙ (μm) | Particle Size, Xᵥ (μm) |
| Control | 2.42 | 7.57 |
| 4.1 | 2.12 | 5.44 |
| 4.2 | 2.12 | 5.61 |
| 4.3 | 2.07 | 5.08 |

Again, by comparing the data of the control sample and jet milled samples, it can be inferred that the jet milling provides significant particle deagglomeration.

### Example 5

### Effect of Jet Milling on Microsphere Residual Moisture Level and Microsphere Morphology

Moisture content of PLGA microspheres was measured by Karl Fischer titration, before and after jet milling. A Brinkman Metrohm 701 KF Titrinio titrator was used, with chloroform-methanol (70:30) as the solvent and Hydranl-Componsite I as the titrant. The PLGA microspheres all were produced by spray drying as described in the introduction portion of the examples, and then jet milled using the conditions shown in Table 9. The grinding pressure was provided by ambient nitrogen at a temperature of approximately 18 to 20° C. The results are shown in Table 10.

TABLE 9
Jet Milling Conditions

| | Injector Gas | Grinding Gas |
|---|---|---|
| Sample | Pressure (bar) | Pressure (bar) |
| 5.1 | 3.6 | 3.1 |
| 5.2 | 1.6 | 1.3 |
| 5.3 | 3.9 | 3.1 |
| 5.4 | 3.0 | 2.9 |

TABLE 10
Effect of Jet Milling on Residual Moisture

| | Pre-Jet Milling | Post-Jet Milling | |
|---|---|---|---|
| | Moisture | Moisture | % Moisture |
| Sample | Level (wt. %) | Level (wt. %) | Reduction |
| 5.1 | 6.62 | 2.18 | 67 |
| 5.2 | 6.62 | 2.32 | 65 |
| 5.3 | 10.28 | 3.19 | 69 |
| 5.4 | 28.60 | 4.20 | 85 |

The data in Table 10 show that a substantial reduction in moisture level occurred. Because moisture levels in excess of 10% can render the powder formulation unstable and not easily handled, jet milling appears to provide a highly useful and unexpected ancillary benefit. That is, along with the deagglomeration, jet milling converted the material into one that is more useable, more stable, and more easily handled.

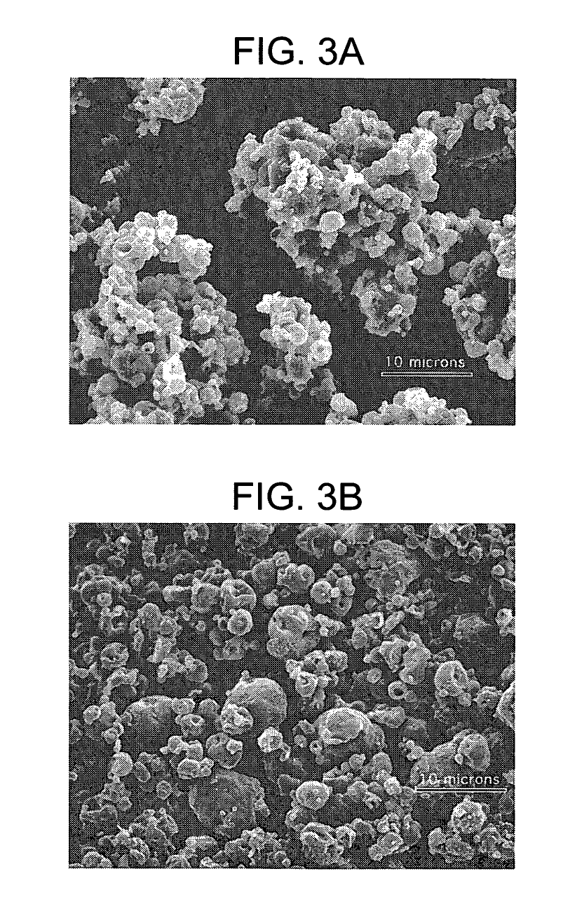

FIGS. 3A-B show SEM images taken before and after jet milling (3.6 bar injection pressure, 3.1 bar grinding pressure, sample 5.1 from Table 9), which indicate that the microsphere morphology remains intact. In particular, FIG. 3A is an SEM of pre-milled microspheres, which clearly shows aggregates of individual particles, while FIG. 3B is an SEM of post-milled microspheres, which do not exhibit similar aggregated clumps. In addition, the overall microsphere structure remains intact, with no signs of milling or fracturing of individual spheres. This indicates that the jet milling is deagglomerating or deaggregating the microparticles, and is not actually fracturing and reducing the size of the individual microparticles.

### Example 6

### Effect of Jet Milling on Blend Residual Moisture Level

Blends were prepared as described in Example 1, and moisture levels were measured as described in Example 5. Table 11 shows the moisture level of the dry blend of microspheres (Lot A), mannitol, and Tween80, as measured before jet milling (control) and after jet milling, with grinding gas at a temperature of 24° C.

TABLE 11
Effect of Jet Milling Parameters on Blend Residual Moisture

| | Moisture Level | Injector Gas | Grinding Gas | % Moisture |
|---|---|---|---|---|
| Sample | (wt. %) | Pressure (bar) | Pressure (bar) | Reduction |
| Control | 2.87 | | | |
| 6.1 | 0.59 | 3.9 | 3.0 | 79 |
| 6.2 | 0.50 | 3.0 | 2.9 | 83 |
| 6.3 | 0.56 | 8.8 | 6.6 | 80 |

The results demonstrate that the moisture content of the dry blended material was reduced by jet milling, by about 80%. Increasing the grinding pressures did not significantly decrease the moisture content further.

### Example 7

### Effect of Jet Milling on Residual Organic Solvent Level

Residual methylene chloride content of PLGA microspheres was measured by gas chromatography before blending and jet milling and then after jet milling. The porous PLGA microspheres (from Lot A described in Example 1) were blended with mannitol at 46 rpm for 30 minutes and then jet milled (injection pressure 3.9 bar, grinding pressure 3.0 bar, and air temperature 24° C.). The assay was run on a Hewlett Packard model 5890 gas chromatograph equipped with a head space autosampler and an electron capture detector. The column used was a DBWax column (30 m×0.25 mm ID, 0.5 μm film thickness). Samples were weighed into a head space vial, which was then heated to 40° C. The head space gas was transferred to the column at a column flowrate of 1.5 mL/min, and then subjected to a 40° C. to 180° C. thermal gradient. The results are shown in Table 12, where parts per million (ppm) is based on the weight of the microspheres.

TABLE 12
Effect of Jet Milling on Residual Organic Solvent

| | Pre-Jet Milling Solvent | Post-Jet Milling Solvent | % Solvent |
|---|---|---|---|
| Sample | Level (ppm) | Level (ppm) | Reduction |
| 7.1 | >557 | 111 | >80 |
| 7.2 | >557 | 150 | >73 |

The results demonstrate that a substantial reduction in the level of residual methylene chloride can be achieved by jet milling the microparticle dry blend formulations.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. A method for making microparticles for use in pharmaceutical formulations, the method comprising:
(a) forming microparticles by a spray drying process which comprises:
spraying an emulsion, solution, or suspension which comprises a solvent and a pharmaceutical agent through an atomizer to form droplets of the solvent and the pharmaceutical agent; and
evaporating at least a portion of the solvent to solidify the droplets and form microparticles; and
(b) jet milling the microparticles to deagglomerate at least a portion of agglomerated microparticles, if any, while substantially maintaining the size and morphology of the individual microparticles.

2. The method of claim 1, wherein the jet milling is performed with a feed gas and/or grinding gas supplied to the jet mill at a temperature of less than about 100° C.

3. The method of claim 2, wherein the temperature is less than about 30° C.

4. The method of claim 1, wherein the feed gas and/or grinding gas supplied to jet mill consists essentially of dry nitrogen gas.

5. The method of claim 1, flurther comprising blending the microparticles with one or more excipients before the jet milling, after the jet milling, or both before and after jet milling the microparticles.

6. The method of claim 1, wherein the emulsion, solution, or suspension further comprises a shell material.

7. The method of claim 6, wherein the shell material is selected from the group consisting of polymers, lipids, sugars, and amino acids.

8. The method of claim 1, wherein the microparticles comprise a shell material surrounding a core of the pharmaceutical agent.

9. The method of claim 1, wherein the microparticles consist essentially of a therapeutic or prophylactic pharmaceutical agent.

10. The method of claim 1, wherein the emulsion, solution, or suspension further comprises a biocompatible polymer.

11. The method of claim 10, wherein the biocompatible polymer is a synthetic polymer selected from the group consisting poly(hydroxy acids), polyanhydrides, poly(ortho)esters, polyurethanes, poly(butyric acid), poly(valeric acid), poly(lactideco-caprolactone), blends and copolymers thereof.

12. The method of claim 1, wherein the microparticles have a number average size between 1 and 20 μm.

13. The method of claim 1, wherein the microparticles have a volume average size between 2 and 50 μm.

14. The method of claim 1, wherein the microparticles have an aerodynamic diameter between 1 and 50 μm.

15. The method of claim 1, wherein the microparticles comprising pharmaceutical agent comprise microspheres having voids or pores therein.

16. The method of claim 1, wherein the pharmaceutical agent is a therapeutic or prophylactic agent.

17. The method of claim 16, wherein the therapeutic or prophylactic agent is selected from the group consisting of non-steroidal anti-inflammatory agents, corticosteroids, antineoplastics, anti-microbial agents, anti-virals, anti-bacterial agents, anti-fungals, antiasthmatics, bronchiodilators, antihistamines, immunosuppressive agents, antianxiety agents, sedatives/hypnotics, antipsychotic agents, anticonvulsants, and calcium channel blockers.

18. The method of claim 16, wherein the therapeutic or prophylactic agent is hydrophobic and the microparticles comprise microspheres having voids or pores therein.

19. The method of claim 16, wherein the therapeutic or prophylactic agent is selected from the group consisting of celecoxib, rofecoxib, docetaxel, paclitaxel, acyclovir, albuterol, alprazolam, amiodaron, amoxicillin, anagrelide, bactrim, beclomethasone dipropionate, biaxin, budesonide, bulsulfan, calcitonin, carbamazepine, ceftazidime, cefprozil, ciprofloxacin, clarithromycin, clozapine, cyclosporine, diazepam, estradiol, etodolac, famciclovir, fenofibrate, fexofenadine, fomoterol, flunisolide, fluticasone propionate, gemcitabine, ganciclovir, , granulocyte colony-stimulating factor, insulin, itraconazole, lamotrigine, leuprolide, loratidine, lorazepam, meloxicam, mesalamine, minocycline, modafinil, mometasone, nabumetone, nelfinavir mesylate, olanzapine, oxcarbazepine, parathyroid hormone-related peptide, phenytoin, progesterone, propfol, ritinavir, salmeterol, sirolimus, SN-38, somatostatin, sulfamethoxazole, sulfasalazine, testosterone, tacrolimus, tiagabine, tizanidine, triamcinolone acetonide, trimethoprim, valsartan, voriconazole, zafirlukast, zilueton, and ziprasidone.

20. The method of claim 1, wherein the pharmaceutical agent is a contrast agent for diagnostic imaging.

21. A pharmaceutical formulation comprising microparticles made by the method of claim 1.

22. A method for making microparticles for use in pharmaceutical formulations, the method comprising:
(a) forming microparticles by a spray drying process which comprises:
spraying an emulsion, solution, or suspension which comprises a solvent and a pharmaceutical agent through an atomizer to form droplets of the solvent and the pharmaceutical agent; and
evaporating at least a portion of the solvent to solidify the droplets and form microparticles;
(b) blending the microparticles with at least one excipient in dry powder form to form a microparticle blend; and
(c) jet milling the microparticles to deagglomerate at least a portion of agglomerated microparticles, if any, while substantially maintaining the size and morphology of the individual microparticles.

23. The method of claim 22, wherein the at least one excipient comprises a surfactant, a sugar, or an amino acid.

24. The method of claim 22, wherein the at least one excipient comprises lactose, mannitol, or trehalose.

25. A pharmaceutical formulation comprising microparticles made by the method of claim 22.
